(19) United States
(12) Patent Application Publication
(10) Pub. No.: US 2023/0368926 A1
(43) Pub. Date: Nov. 16, 2023

(54) INTEROPERABLE PRIVACY-PRESERVING MULTI-CENTER DISTRIBUTED MACHINE LEARNING METHOD FOR HEALTHCARE APPLICATIONS

(71) Applicant: Cipherome, Inc., San Jose, CA (US)

(72) Inventors: Samuel Kim, La Palma, CA (US); Min Sang Kim, Sunnyvale, CA (US); Mijeong Ban, San Jose, CA (US)

(21) Appl. No.: 18/314,692

(22) Filed: May 9, 2023

Related U.S. Application Data
(60) Provisional application No. 63/341,936, filed on May 13, 2022.

(51) Int. Cl. G16H 50/70; G16H 10/60; G16H 50/20

(52) U.S. Cl. CPC G16H 50/70; G16H 10/60; G16H 50/20

(57) ABSTRACT

A learning system deploys a dynamic data conversion module (DDCM) that is customized to perform one or more extract, transform, and load (ETL) operations on the local client data that is used to train at least a portion of a master machine-learning model for the distributed learning framework. The DDCM envelopes a set of components including at least a pre-assessment toolkit and a ETL model. The pre-assessment toolkit is configured to collect statistics and abstract information from the client database of the client device and provide the statistics and abstract information to the learning system. Based on the statistics and abstract information of a respective client device, the learning system generates conversion logics and standardized vocabularies and provides them to the DDCM of the client device.

### CROSS-REFERENCE TO RELATED APPLICATIONS

This application claims the benefit of U.S. Provisional Application No. 63/341,936, filed on May 13, 2022, which is incorporated herein in its entirety.

### TECHNICAL FIELD

This disclosure relates generally to artificial intelligence (AI) and machine-learning (ML) applications, and particularly to federated learning in the context of electronic health records.

### BACKGROUND

Healthcare data are typically fragmented and private. Different medical institutes own their own electronic health records of patients, and the data are difficult to share across institutes because of privacy concerns. Federated learning allows multiple data owners to collaborate with each other to train a master machine-learning model without exposing individual datasets that have privacy concerns. Federated learning can be adapted for application to healthcare data to mitigate issues with distributed machine learning approaches where different medical institutes share models and/or anonymized data rather than raw data, while solving technical issues such as consolidating derivatives from various client devices, imbalance in data, network latency and security. However, there is technical difficulty in coordinating the learning process across multiple client devices.

### SUMMARY

A learning system deploys, to one or more client devices, modules to be deployed in a learning environment of a respective client device. The learning environment of a respective client device may include modules for the client device (or client node) to collaborate with the central learning system and other client devices via a distributed learning (e.g., federated learning, split learning) framework. In one embodiment, the modules deployed by the learning system can be used to dynamically execute ETL operations on the local client data, participate in training a machine-learning model based on the client data, and perform one or more inference tasks in conjunction with applications on the client device

In one embodiment, the learning system deploys a dynamic data conversion module (DDCM) that is customized to perform one or more extract, transform, and load (ETL) operations on the local client data that is used to train at least a portion of a master machine-learning model for the distributed learning framework. Specifically, the DDCM envelopes a set of components including at least a pre-assessment toolkit and a ETL model. The pre-assessment toolkit is configured to collect statistics and abstract information from the client database of the client device and provide the statistics and abstract information to the learning system. Based on the statistics and abstract information of a respective client device, the learning system generates conversion logics and standardized vocabularies and provides them to the DDCM of the client device.

The ETL model of a respective client device is configured to receive the conversion logic and standardized vocabularies from the learning system and execute one or more ETL operations to convert the client data to a common data model. In particular, the conversion logic is tailored to the local client data in that the executed ETL operations convert the particular data fields and local vocabularies of the local client data to target schema compatible with the machine-learning model being trained. Therefore, updates to the ETL model at one client device may differ from the ETL model deployed at another client device that encodes the client data differently with a different set of fields and vocabularies.

In one embodiment, the learning system may also deploy a dynamic data learning module (DDLM) to the client devices that are configured to train a machine-learning model in a distributed manner in collaboration with other client devices without exposure to client data. The DDLM is configured to extract converted features from the common data model and train a local model based on the converted values in the training data. The local model may be trained based on data in the target schema. The DDLM may coordinate with the learning system to share parameters of the local model and receive parameters of the master model, so that the client device has access to the master model without compromising the privacy of the client data.

In a distributed learning framework in which client data may be prevented from being shared due to privacy concerns, it may be technically difficult and resource-consuming for a client node (e.g., medical clinic, hospital) to perform ETL operations, such that data is in target schema compatible with the master machine-learning model (and thus, the local model to be trained at the client). By injecting the customized conversion logic into the ETL model of a learning environment of a client device without exposing the client data of the client, the learning system allows many client devices to effectively participate in the distributed learning framework without having to manually configure the ETL operations internally.

Moreover, features may frequently get newly added, deleted, or otherwise modified, as healthcare data and features related to the data quickly develop over time due to, for example, ongoing research. In these instances, the learning system can dynamically generate new conversion logic that corresponds to the updated features and deploy the updated conversion logic to the ETL models of the client devices. In this manner, as client nodes dynamically develop different types of data and features related to the data, this information can be quickly and effectively reflected into the machine-learning model.

### BRIEF DESCRIPTION OF DRAWINGS

FIG. 1 illustrates a system environment for interoperable distributed learning, according to one embodiment.

FIG. 2 illustrates a block diagram of an architecture of a learning environment of a client device, according to one embodiment.

FIG. 3 illustrates a method of performing dynamic data conversion using DDCM, according to one embodiment.

FIG. 4 is an example flowchart for deploying a DDCM for performing ETL operations at a client node, according to one embodiment.

The figures depict various embodiments of the present disclosure for purposes of illustration only. One skilled in the art will readily recognize from the following discussion that alternative embodiments of the structures and methods illustrated herein may be employed without departing from the principles of the disclosure described herein.

### DETAILED DESCRIPTION

The Figures (FIGS.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

Disclosed is a configuration (including a system, a process, as well as a non-transitory computer readable storage medium storing program code) for generating a dynamic data conversion module and deploying the dynamic data conversion module on one or more client nodes in a learning environment.

### Overview

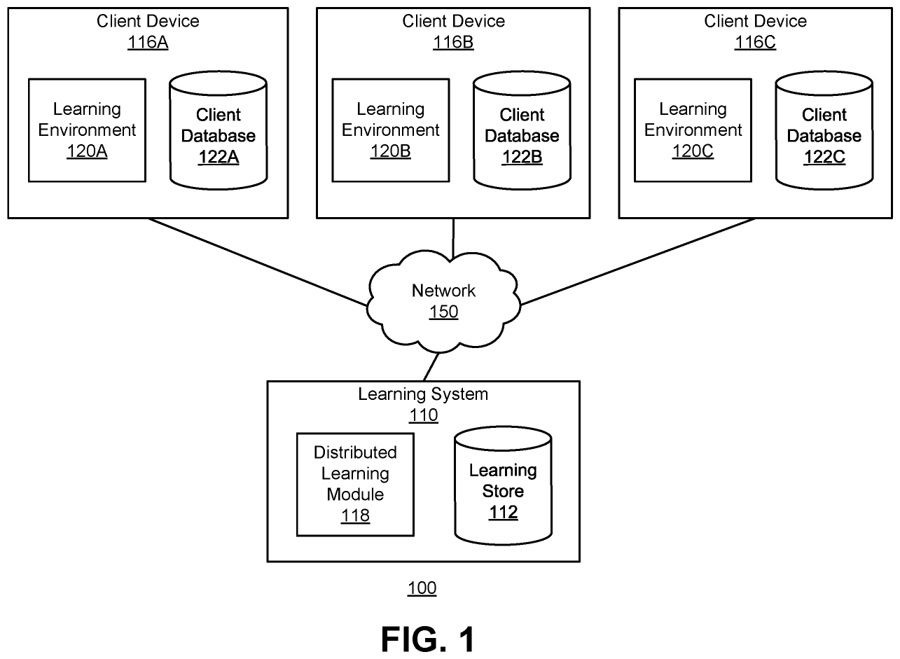

FIG. 1 illustrates a system environment 100 for interoperable distributed learning, according to one embodiment. The system environment 100 shown in FIG. 1 comprises one or more client nodes 116A, 116B, and 116C, a learning system 110, and a network 150. The learning system 110 in one embodiment also includes a distributed learning module 118 and a learning store 112. In alternative configurations, different and/or additional components may be included in the system environment 100 and embodiments are not limited hereto.

The learning system 110 (e.g., the distributed learning module 118) deploys, to one or more client nodes 116, modules to be deployed in a learning environment of a respective client device. In one embodiment, learning system 110 described herein may allow the client nodes 116 to perform distributed machine learning without sharing privacy-sensitive information that are stored and managed in respective client nodes. In other words, the learning system 110 may enable a distributed learning framework that is interoperable, yet privacy-preserving so that participating institutes (e.g., corresponding to the client nodes) perform various research using not only their own data but also other institutes' data without compromising privacy of the other institutes' data.

In one embodiment, the distributed machine learning framework orchestrated by the learning system 110 is responsible for training a master machine-learning model used for a variety of inference tasks, for example, health event classification, natural language processing (NLP), image processing, video processing, or audio processing applications in the context of, for example, healthcare applications. The machine-learning model is trained across one of more client nodes 116, and may be configured as artificial neural networks (ANNs), recurrent neural networks (RNNs), deep learning neural networks (DNNs), bi-directional neural networks, transformers, classifiers such as support vector machines (SVMs), decision trees, regression models such as logistic regression, linear regression, stepwise regression, generative models such as transformer-based architectures including GPT, BERT, encoder-decoders, or variational autoencoders, and the like.

In one embodiment, the learning system 110 deploys a distributed learning framework that is a federated learning method. In such a method, each client node 116 trains a local copy of a master machine learning model using client data at the client node (that cannot be exposed to other client nodes 116 and/or the learning system 110), and parameters of the master machine learning model are determined by aggregating the trained local models obtained across the set of client nodes 116. In another embodiment, the distributed learning framework is a split learning method in which a client node 116 may train a dedicated section of the machine learning model, and the different portions of the machine-learning model are aggregated by the learning system 110. While the remainder of this specification is primarily described with respect to a federated learning or split learning framework, it is appreciated that the embodiments herein may be applied to any type of distributed machine learning method that orchestrates the training process of machine learning models across a set of decentralized client nodes.

In particular, a significant difficulty for distributed machine learning using privacy-sensitive health or medical data is that data may generally be fragmented and private for each institution, as different health and medical institutes locally store their own electronic health records of patients, and the data are difficult to share across institutes because of privacy concerns. Moreover, each institution may store the data according to their own data schema and may be heterogenous with respect to the types of fields, data distribution, and the like. For example, one institution may classify diseases according to the International Statistical Classification of Diseases (ICD-10) while another institution may classify diseases according to Korean Standard Classification of Diseases (KCD5). It is difficult to orchestrate training using a distributed learning system across different client nodes when institutions locally store and manage data according to their own data schema, and the machine learning models have to be trained using a standardized set of input data, features, and labels.

Thus, in one embodiment, the learning system 110 described herein provides modules for deployment in a learning environment of a respective client node 116 that allow the client node 116 to collaborate with the learning system 110 and other client nodes 116 via a distributed learning (e.g., federated learning, split learning) framework. In one embodiment, the modules deployed by the learning system 110 can be used by the client node 116 to dynamically execute ETL operations on local client data, participate in training a master machine-learning model based on the client data, and perform one or more inference tasks in conjunction with applications on the client node 116.

In one embodiment, the learning system 110 deploys a dynamic data conversion module (DDCM) that is customized to perform one or more extract, transform, and load (ETL) operations on the local client data for training least a portion of a master machine-learning model within the distributed learning framework. Specifically, the DDCM envelopes a set of components including at least a pre-assessment toolkit and a ETL model that performs one or more ETL operations on client data to a target schema. In one instance, the pre-assessment toolkit is configured to collect statistics and abstract information from the client database of the client device and provide the statistics and abstract information to the learning system 110. Based on the statistics and abstract information of a respective client device, the learning system 110 generates conversion logics and standardized vocabularies and provides them to the DDCM of the client node 116.

The ETL model of a respective client node 116 is configured to receive the conversion logic and standardized vocabularies from the learning system and execute one or more ETL operations to convert the client data to a common data model. In particular, the conversion logic is tailored to the local client data in that the executed ETL operations convert the particular data fields and local vocabularies of the local client data to target schema compatible with the machine-learning model being trained. Therefore, updates to the ETL model at one client node 116 may differ from the ETL model deployed at another client node 116 that encodes the client data differently with a different set of fields and vocabularies.

In one embodiment, the learning system 110 may also deploy a dynamic data learning module (DDLM) to the client nodes 116 that are configured to train a machine-learning model in a distributed manner in collaboration with other client nodes 116 without exposing the client data to other client nodes 116 (e.g., other institutions) and/or the learning system 110. The DDLM is configured to extract converted features from the common data model and train a local model based on the converted values in the training data. The local model may be trained based on data in target schema. The DDLM may coordinate with the learning system 110 to share parameters of the local model and receive parameters of the master model, so that the client node 116 has access to the master model without compromising the privacy of the client data.

In a distributed learning framework in which client data may be prevented from being shared due to privacy concerns, it may be technically difficult and resource-consuming for a client node 116 (e.g., medical clinic, hospital) to perform ETL operations, such that data is in target schema compatible with the master machine-learning model (and thus, the local model to be trained at the client). By injecting the customized conversion logic into the ETL model of a learning environment of a client node 116 without exposing the client data, the learning system 110 allows many client devices to effectively participate in the distributed learning framework without having to manually configure the ETL operations internally.

Moreover, features may frequently get newly added, deleted, or otherwise modified, as healthcare data and features related to the data quickly develop over time due to, for example, ongoing research. In these instances, the learning system 110 can dynamically generate updated conversion logic that corresponds to the updated features and deploy the updated conversion logic to the ETL models of the client nodes 116. In this manner, as different clients dynamically develop different types of data and features related to the data, this information can be quickly and effectively reflected into the machine-learning model without manually configuring the ETL operations at the client nodes 116 themselves.

The client nodes 116 may each correspond to a computing system (e.g., server) that manages health-related data for a respective institution or organization, such as a hospital, medical research facility, etc. Thus, data stored for a client node 116 may be privacy-sensitive in that it includes electronic health or medical records for patients or subjects and may be subject to compliance with privacy laws and regulations (e.g., Health Insurance Portability and Accountability Act (HIPAA)) for protecting sensitive information. For example, client node 116A in the system environment 100 of FIG. 1 may correspond to a relatively small-sized clinic, client node 116B may correspond to a bio-medical research facility in a different geographical region than the clinic, and client node 116C may correspond to a large hospital.

The client data of a respective client node 116 may have one or more fields (e.g., patient ID, patient name, HAS-BLED scores) and values for those fields. The client data may be owned by an entity of the client node 116 in the sense that the entity may have control over exposure of the data, whether the data can be shared or accessed by other entities, and the like. The client data may be stored locally (e.g., on a local server, a private cloud, a disk on the client node 116) or may be stored on a cloud platform in a remote object datastore. As described above, each client node 116 may store their data according to a particular data schema. Therefore, while the client nodes 116 may collectively record information that describes the same or similar property (e.g., BMI) with one another, the way one institution encodes data may differ from the way another institution encodes the same or similar data with respect to the types of fields collected, vocabularies, categorization schemes, and the like.

In one embodiment, a client node 116 receives one or more modules from the learning system 110 to deploy a learning environment that allows the entity of the client node 116 to participate in a distributed learning framework and contribute to training a master machine-learning model based on client data stored with respect to the entity, without the need to expose the actual data to other participants. The client node 116 receives a custom DDCM from the learning system 110 that allows one or more ETL operations to be performed on the client data. The client node 116 receives a DDLM from the learning system 110 that allows the client node 116 to train a local copy or a local portion of the master model based on the converted data, share parameters of the local model to the learning system 110, and/or receive a set of parameters corresponding to the master machine-learning model that was aggregated by the learning system 110 across the set of participants. The aggregated master model is stored in the learning store 112.

While three example client nodes 116A, 116B, 116C are illustrated in the system environment 100 of FIG. 1, in practice many client nodes 116 may communicate with the systems in the environment 100. In one embodiment, a client node 116 is a conventional computer system, such as a desktop or laptop computer. Alternatively, a client node 116 may be a device having computer functionality, such as a personal digital assistant (PDA), a mobile telephone, a smartphone or another suitable device. A client node 116 is configured to communicate via the network 150.

The client nodes 116 and the learning system 110 are configured to communicate via the network 150, which may comprise any combination of local area and/or wide area networks, using both wired and/or wireless communication systems. In one embodiment, the network 150 uses standard communications technologies and/or protocols. For example, network 150 includes communication links using technologies such as Ethernet, 802.11, worldwide inter-operability for microwave access (WiMAX), 3G, 4G, code division multiple access (CDMA), digital subscriber line (DSL), etc. Examples of networking protocols used for communicating via the network 150 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 150 may be represented using any suitable format, such as hypertext markup language (HTML) or extensible markup language (XML). In some embodiments, all or some of the communication links of the network 150 may be encrypted using any suitable technique or techniques.

### Learning Environment of Client Node

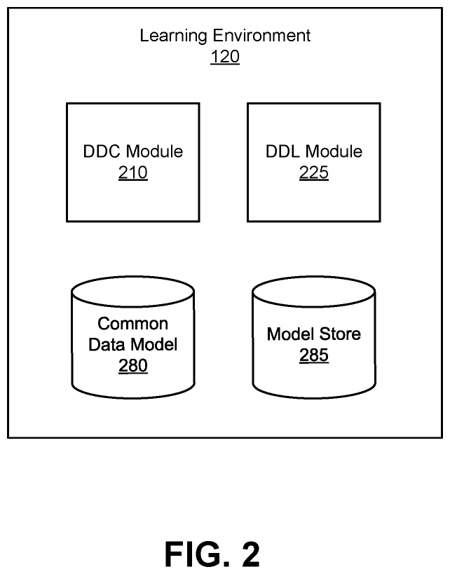

FIG. 2 illustrates a block diagram of an architecture of a learning environment 120 of a client node 116, according to one embodiment. The learning environment 120 shown in FIG. 2 includes a DDC module 210 and a DDL module 225. The learning environment 120 also includes a common data model datastore 280 and a model datastore 285. In other embodiments, the learning environment 120 may include additional, fewer, or different components for various applications. Conventional components such as network interfaces, security functions, load balancers, failover servers, management and network operations consoles, and the like are not shown so as to not obscure the details of the system architecture. Moreover, while not shown in FIG. 2, the learning environment 120 may have access to one or more databases managed by an entity of the client node 116.

The DDCM 210 may be deployed in the learning environment 120 of a client node 116 from the distributed learning module 118 of the learning system 110. In one embodiment, the DDCM 210 includes data pre-assessment toolkits and an ETL model. Responsive to receiving a request from the distributed learning module 118, the DDCM 210 executes a data pre-assessment using the pre-assessment toolkits on the client database. The DDCM 210 collects the statistics and abstract information from the client databases describing particular properties of the data. The DDCM 210 provides the collected statistics and abstract information to the distributed learning module 118.

In one instance, the statistics and abstract information collected by the DDCM 210 includes metadata like a list of table identifiers used to store the data, the number of fields in each table, or a number of records in the table. As another example, the metadata may include information on fields, such as unique values of a field or frequency distribution (e.g., range, min-max, average) of values for a field. The statistics and abstract information may also include which medical code system (e.g., ICD-10, KCD5) an entity (e.g., hospital, clinic, research facility) managing the client data encodes the data in, and a list of unique medical codes present in the client data. Thus, the statistics and abstract information may differ from one client node 116 to another, depending on the data schema used.

The DDCM 210 receives, from the distributed learning module 118, updated conversion logic and standardized vocabularies for application to the ETL model. The standardized vocabularies represent a set of vocabularies for encoding values for the data fields that should be used across the participating client nodes 116, since the master model will be trained based on the standardized vocabulary of values. The conversion logic is customized logic tailored to the client data of a respective client node 116 that includes instructions for performing one or more ETL operations to convert the client data to a target schema compatible with the local model to be trained. For example, the conversion logic may include logic for data field conversion that collects a set of fields and converts the fields into a target schema in the common data model store 280. As another example, the conversion logic may include conversion of a system of measurements (e.g., imperial to metric) or value bucketing conversions. As another example, the conversion logic may also include a set of mappings in the form of, for example, a mapping table (e.g., CSV file) that maps local vocabularies (e.g., local medical codes) to standardized medical codes (e.g., international medical codes) listed in the standardized vocabulary.

The DDCM 210 executes ETL operations to convert the client data to the common data model 280. In particular, the DDCM 210 extracts the client data from one or more data sources. The DDCM 210 transforms the data based on the conversion logic and standardized vocabulary received from the distributed learning module 118. The DDCM 210 loads the converted data to the common data model store 280, such that the converted data can be used to train the local model.

The DDLM 225 may also be deployed in the learning environment 120 of the client node 116 from the distributed learning module 118 of the learning system 110. In one embodiment, the DDLM 225 includes a base machine-learning model and software for data extraction and local model training. Specifically, the base machine-learning model may be a local copy of the master machine-learning model or at least a portion of the master model, in which parameters of the base model are not determined yet or have to be updated to reflect new updates in the training data. The DDLM 225 performs a training process to train the local model, such that the local model can be aggregated across the client nodes 116. The architecture of the local model may be used to perform any type of inference or generation tasks, including, but not limited to adverse drug reaction (ADR) predictions, cancer detection from images, cancer type classification, drug-to-drug interactions, disease prediction, patient stratification during clinical trials, drug efficacy predictions, and the like.

The DDLM 225 extracts a set of training data from the common data model 280 of the client node 116. The training data includes one or more instances of training inputs and outputs. The training inputs and outputs may have been converted from an original data source by the ETL operations performed by the DDCM 210. The DDLM 225 trains the local model with the extracted data. In one embodiment, the DDLM 225 may train the local model by repeatedly performing a forward pass step, a loss function determination step, and a backpropagation step. During the forward pass step, the training inputs are propagated through the local model to generate estimated outputs. During the loss function determination step, a loss function that indicates a difference (e.g., L2, L1 norm) between the estimated outputs and the corresponding training outputs is determined. During the backpropagation step, error terms from the loss function are used to update the parameters of the local model. These steps may be repeated multiple times using for example, different training batches, until a convergence criterion is reached. The DDLM 225 provides the distributed learning module 118 with the local model such that a consolidated master model can be generated.

In one embodiment, after the consolidation is performed across the client nodes 116, the DDLM 225 receives parameters of the consolidated master model from the distributed learning module 118. In this manner, the client node 116 can use the updated master model trained based on local data from other participating client nodes 116 to perform various types of health-related tasks (e.g., ADR predictions, disease diagnosis, etc.) without compromising the privacy of the local data from the other client nodes 116. For example, a master model for predicting ADR of patients may be provided to an institution that by itself, does not store sufficient data for training the ADR prediction model. The institution applies the master model to generate predictions for ADR for its patients without having to manually coordinate access to privacy-sensitive data of other institutions for training the ADR prediction model.

### Detailed System for Performing Distributed Learning Using Dynamic Data Conversion

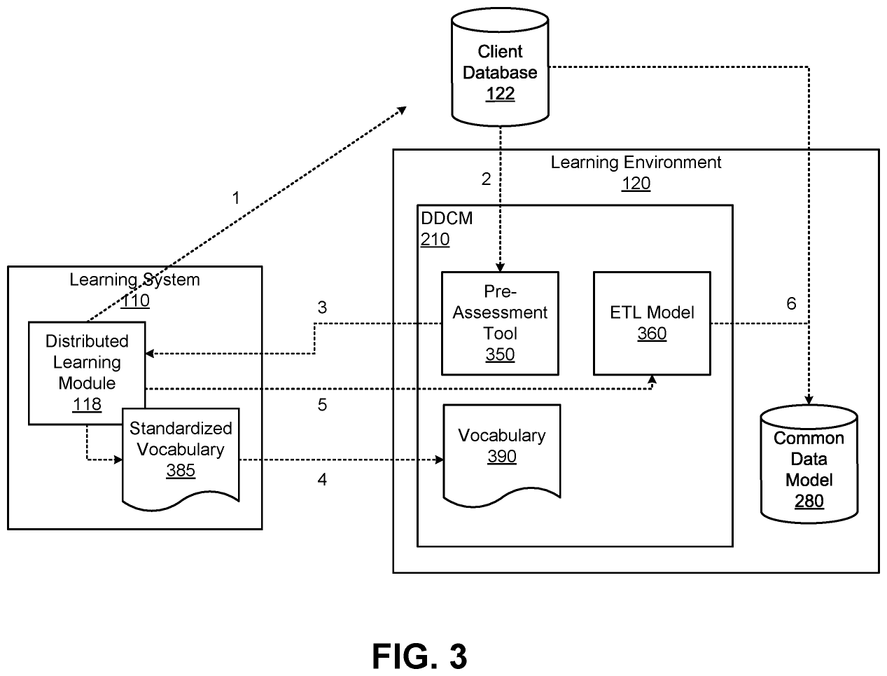

FIG. 3 illustrates a method of performing dynamic data conversion using DDCM 210, according to one embodiment. The detailed system diagram shown in FIG. 3 includes the learning system 110 (also including the distributed learning module 118), the learning environment 120 of a client node 116, and a client database store 122. The DDCM 210 further includes pre-assessment toolkit 350, an ETL model 360, and vocabulary store 390.

In a multi-center environment, the distributed learning module 118 deploys an initial version of the DDCM 210 to a particular client node 116 (step “1”). The DDCM 210 includes a pre-assessment toolkit 350 and a base ETL model 360. The base ETL model 360 may include a set of initial conversion logic for converting client data to common data model 280. The distributed learning module 118 initiates a data pre-assessment process on the local client data through the pre-assessment toolkit 350. As described in conjunction with FIG. 2, the pre-assessment toolkit 350 collects statistics and abstract information about the client data (step “2”), and sends the information to the distributed learning module 118 (step “3”).

The distributed learning module 118 collects the local vocabularies 390 in each client node 116. The distributed learning module 118 also maintains a standardized set of vocabularies 385 that conform with target schema for training the machine-learning model. For each client node 116, the distributed learning module 118 maps the local vocabularies (e.g., KCD5) to standardized vocabularies (e.g., ICD-10) to generate a vocabulary mapping that maps one or more local vocabularies to one or more standardized vocabularies. The distributed learning module 118 deploys the standardized vocabularies as well as the vocabulary mapping for the client node 116 to the client node 116 (step “4”). Thus, the DDCM 210 of client nodes 116 that use different local vocabularies may receive different vocabulary mappings.

The distributed learning module 118 also updates the base ETL model 360 at the DDCM 210 of the client nodes 116 so that a respective ETL model 360 converts the client data to the common data model 280 accurately (step “5”). For example, the central server may update the ETL module with conversion logics described in conjunction with FIG. 2 that specify instructions for filtering, deduping, coalescing, joining, transforming, and mapping data fields and vocabularies. As described in FIG. 2, conversion logics may include data field conversion, conversion of a system of measurements or value bucketing conversions, mapping of local vocabularies to standardized vocabularies, among other types of operations.

As an example, one institution could have basic information of a patient (e.g., first name and last name, age, sex, or ethnicity) spread out in different tables or files with different field names from the target schema. The logic for data field conversion may be configured to convert data fields from client data at the client node 116 to corresponding data fields of the target schema. For example, value for a data field “*31-0.0” of UK Biobank indicating a sex of a participant at a client data source may be converted to corresponding values for data fields “gender concept ID,” “gender source value,” and “gender source concept ID” for the target schema. As yet another example, the value for a data field “*34-0.0” of UK Biobank indicating year of birth of participant at the source may be converted to corresponding values for data fields “year of birth” for the target schema.

As another example, logic for conversion of a system of measurements may be configured to convert data values in kilogram to pounds for weight, centimeter to feet for height, or cubic centimeter to milliliters for volume, thus, convert one unit of measurement to another unit of measurement. As yet another example, logic for value bucketing conversions may be configured to transform a set of values categorized according to one bucketing method to another set of values categorized according to another bucketing method. Specifically, different institutions that correspond to different client nodes may categorize values differently. For example, for body mass index (BMI) measurements, one hospital associated with a client node 116 may set the bucketing as the following:

TABLE 1
Example bucket values for BMI for a client
node associated with a hospital.

| BMI Value | Bucket Value |
|---|---|
| <18.5 | Underweight |
| 18.5-24.9 | Normal |
| 25.0-29.9 | Overweight |
| >=30.0 | Obese |

However, another hospital associated with another client node 116 may set the bucketing as the following:

TABLE 2
Example bucket values for BMI for another client
node associated with a second hospital.

| BMI Value | Bucket Value |
|---|---|
| <25 | 0 |
| >=25 | 1 |

Therefore, logic for value bucketing conversion may transform a set of categorization values for a first bucketing method (e.g., Table 1) to another set of categorization values for a second bucketing method (e.g., Table 2). For example, the values “Underweight” and “Normal” in the BMI field may be converted to 0, and the values “Overweight” and “Obese” in the BMI field may be converted to 1.

As yet another example, the CHADS2 score is a score for predicting the risk of thromboembolic stroke in patients and HAS-BLED scores are used to predict bleeding risk in patients with atrial fibrillation. The factors for assessing CHADS2 scores is based on congestive heart failure, hypertension, whether age of the subject is 75 years or older, diabetes mellitus, and presence of previous strokes or transient ischaemic attacks. On the other hand, the factors for assessing HAS-BLED scores use multiple or raw values based on hypertension, abnormal renal or liver function, stroke, bleeding history or predisposition, labile international normalized ratio, whether the subject is an elderly over 65 years of age, and drugs/alcohol concomitantly score (procedure). Thus, CHADS2 scores bucket the age value into >=75, <75, while the HAS-BLED scores bucket the age value into <=65, >65 while evaluating the scores. Therefore, logic for value bucketing conversions may standardize the bucketing system for age to one standardized bucketing system (e.g., <=65, >65) to assess HAS-BLED scores.

As yet another example, logic for data distribution conversions may be configured to obtain statistics on values for a particular field (e.g., height, weight, BMI) of client data without exposing the actual values, and convert the values to fit to a different data distribution. For example, an institution may store height in actual values of centimeters. The distributed learning module 118 collects statistics (e.g., mean and standard deviation) on the height values (e.g., from pre-assessment toolkit 350), and generate conversion logic for normalizing the height values in the client data to a normalized Gaussian distribution.

Based on the updated conversion logic and vocabulary mappings, the ETL model 360 executes one or more ETL operations on the client data to generate converted data in the common data model datastore 280 (step “6”). In particular, the ETL model 360 extracts data from the appropriate sources and applies the injected conversion logic to the extracted data. The ETL model 360 stores the converted data in the common data model datastore 280. For example, for a data conversion logic to join one or more data fields (e.g., name of patient and patient identifier) in the client data together to create a joined data field, the ETL model 360 may extract the fields from the appropriate data sources, combine the values for the fields and create a joined field in the common data model store 280. As another example, for a conversion logic to convert a system of measurements, the ETL model 360 may identify fields in the client data that use one unit of measurement (e.g., cm), generate new fields that convert the values to another unit (e.g., feet) compatible with the target schema and store the fields in the common data model store 280. As yet another example, for a conversion logic to map local vocabularies (e.g., ICPC-2) to standardized vocabularies (e.g., ICD-10) the ETL model 360 obtains the vocabulary mapping that was received from the distributed learning module 118 and transforms vocabularies in the client data to the standardized vocabularies according to the mapping.

Moreover, while the specification describes a process of generating updated conversion logic and executing the ETL model, it is appreciated that as the data fields or the properties of the client data change over time, for example, over the course of data research. Thus, the pre-assessment toolkit 350 may continuously monitor the client data, and send updated statistics and abstract information as the properties of the client data change over time. The distributed learning module 118 may continuously update the set of conversion logics to reflect the changes in the client data, and provide updated conversion logics to the ETL model 360. The ETL model 360 may in turn perform updated ETL operations reflecting the updated conversion logic and store the converted values in the common data model store 280.

Although not shown in FIG. 3, as described in conjunction with FIG. 2, the DDLM 225 extracts training data from the common data model store 280. In particular, the training data includes converted data in the target schema. The DDLM 225 trains the local model to iteratively update parameters based on the training inputs and outputs in the training data, as described in conjunction with FIG. 2. The DDLM 225 provides parameters of the local model to the distributed learning module 118 of the learning system 110.

The distributed learning module 118 aggregates local models across the one or more client nodes 116 and coordinates training of the master machine-learning model. In one embodiment, when the distributed learning framework is federated learning, the distributed learning module 118 may aggregate the local copies of the model across the client nodes 116 via an average, weighted average, or another statistical function. In another embodiment, when the distributed learning framework is split learning, the distributed learning module 118 may aggregate the various portions of the model split across the client nodes 116 to form the master model. In one instance, the distributed learning module 118 provides the parameters of the master model to each of the client nodes 116, such that the DDLM 225 at each client node 116 is able to perform inference or generation tasks using the master model.

Similar to that described above, the DDLM 225 may also continuously monitor the common data model store 280 to receive indications of updated conversion data. The DDLM 225 incorporates the updated data in training the local model when such an indication is received. In this manner, the DDCM 210 in conjunction with the DDLM 225 of the client node 116 allows the entity to quickly and efficiently incorporate the distributed learning framework even if changes are frequently made to the client data.

### Method for Deploying a DDCM for Performing ETL Operations

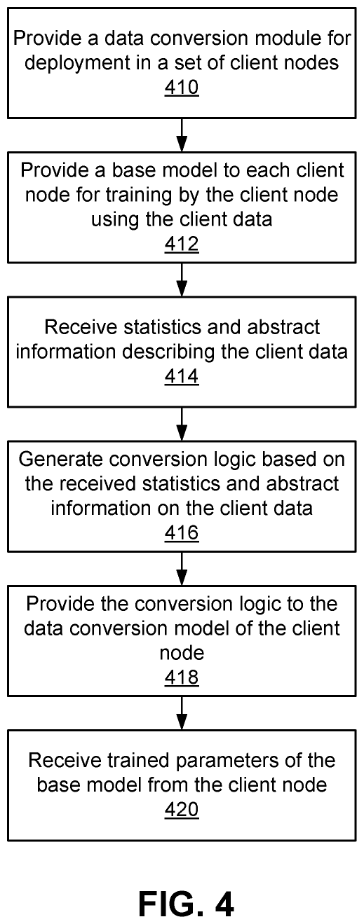

FIG. 4 is an example flowchart for deploying a DDCM for performing ETL operations at a client node, according to one embodiment. The learning system 110 provides 410 a data conversion module for deployment in a set of client nodes. The data conversion module when installed on client node may be configured to generate a set of components. The set of components may include at least a pre-assessment tool and a data conversion model for performing one or more conversion operations on client data associated with the client node. The learning system 110 provides 412, to each client node, a base model to the client node for training by the client node using the client data.

The learning system 110 receives 414, from the pre-assessment tool on each client node, statistics and abstract information describing the client data for the client node. The learning system 110 generates 416, for each client node, conversion logic based on the received statistics and abstract information on the client data for the client node. The conversion logic may include instructions for converting values of the client data for one or more data fields to match a target schema for training the base model. The learning system 110 provides 418, to each client node, the conversion logic to the data conversion model of the client node. The learning system 110 receives 420, from each client node, trained parameters of the base model from the client node. The parameters of the base model may be trained using converted values by executing the data conversion model to perform one or more conversion operations using the conversion logic.

### SUMMARY

The foregoing description of the embodiments of the disclosed configuration has been presented for the purpose of illustration; it is not intended to be exhaustive or to limit the disclosed configuration to the precise forms disclosed. Persons skilled in the relevant art can appreciate that many modifications and variations are possible in light of the above disclosure.

Some portions of this description describe the embodiments of the disclosed configuration in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments of the disclosed configuration may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments of the disclosed configuration may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the disclosed configuration be limited not by this detailed description, but rather by any claims that issue on an application based hereon. Accordingly, the disclosure of the embodiments of the disclosed configuration is intended to be illustrative, but not limiting, of the scope of the disclosed configuration, which is set forth in the following claims.

What is claimed is:

1. A method, comprising:
providing a data conversion module for deployment in a set of client nodes, the data conversion module when installed on client node configured to generate a set of components, the set of components including at least a pre-assessment tool and a data conversion model for performing one or more conversion operations on client data associated with the client node;
providing, to each client node, a base model to the client node for training by the client node using the client data;
receiving, from the pre-assessment tool on each client node, statistics and abstract information describing the client data for the client node;
generating, for each client node, conversion logic based on the received statistics and abstract information on the client data for the client node, wherein the conversion logic includes instructions for converting values of the client data for one or more data fields to match a target schema for training the base model;
providing, to each client node, the conversion logic to the data conversion model of the client node; and
receiving, from each client node, trained parameters of the base model from the client node, wherein the parameters of the base model are trained using converted values by executing the data conversion model to perform one or more conversion operations using the conversion logic.

2. The method of claim 1, wherein conversion logic generated for a first client node in the set of client nodes is different from conversion logic generated for a second client node.

3. The method of claim 1, wherein the conversion logic includes logic for one or a combination of data field conversion, a conversion of measurements, a value bucketing conversion, and vocabulary conversion from a first vocabulary to a second vocabulary.

4. The method of claim 1, the statistics and abstract information received from a client node including one or a combination of metadata on data fields of the client data or statistics on values of the client data, without exposing actual values of the client data.

5. The method of claim 1, the statistics and abstract information for a client node including local vocabulary used to encode one or more values in the client data, and the method further comprising:
generating a mapping from the local vocabulary to a standardized vocabulary; and
providing the mapping to the data conversion module.

6. The method of claim 1, further comprising:
receiving, from a client node, an indication that the statistics and abstract information for the client data has been updated;
generating updated conversion logic reflecting the changes to the statistics and abstract information; and
providing the updated conversion logic to the data conversion module of the client node.

7. The method of claim 1, further comprising combining the trained parameters of the base models from the set of client nodes to form a trained master model.

8. A non-transitory computer readable medium comprising stored instructions, the stored instructions when executed by at least one processor of one or more computing devices, cause the one or more computing devices to:
provide a data conversion module for deployment in a set of client nodes, the data conversion module when installed on client node configured to generate a set of components, the set of components including at least a pre-assessment tool and a data conversion model for performing one or more conversion operations on client data associated with the client node;
provide, to each client node, a base model to the client node for training by the client node using the client data;
receive, from the pre-assessment tool on each client node, statistics and abstract information describing the client data for the client node;
generate, for each client node, conversion logic based on the received statistics and abstract information on the client data for the client node, wherein the conversion logic includes instructions for converting values of the client data for one or more data fields to match a target schema for training the base model;
provide, to each client node, the conversion logic to the data conversion model of the client node; and
receive, from each client node, trained parameters of the base model from the client node, wherein the parameters of the base model are trained using converted values by executing the data conversion model to perform one or more conversion operations using the conversion logic.

9. The non-transitory computer readable medium of claim 8, wherein conversion logic generated for a first client node in the set of client nodes is different from conversion logic generated for a second client node.

10. The non-transitory computer readable medium of claim 8, wherein the conversion logic includes logic for one or a combination of data field conversion, a conversion of measurements, a value bucketing conversion, and vocabulary conversion from a first vocabulary to a second vocabulary.

11. The non-transitory computer readable medium of claim 8, the statistics and abstract information received from a client node including one or a combination of metadata on data fields of the client data or statistics on values of the client data, without exposing actual values of the client data.

12. The non-transitory computer readable medium of claim 8, the statistics and abstract information for a client node including local vocabulary used to encode one or more values in the client data, and the instructions when executed by the computing devices further causing the computing devices to:
generate a mapping from the local vocabulary to a standardized vocabulary; and
provide the mapping to the data conversion module.

13. The non-transitory computer readable medium of claim 8, the instructions when executed by the one or more computing devices causing the computing devices to:
receive, from a client node, an indication that the statistics and abstract information for the client data has been updated;
generate updated conversion logic reflecting the changes to the statistics and abstract information; and
provide the updated conversion logic to the data conversion module of the client node.

14. The non-transitory computer readable medium of claim 8, the instructions when executed by the one or more computing devices causing the computing devices to combine the trained parameters of the base models from the set of client nodes to form a trained master model.

15. A computer system comprising:
one or more computer processors; and
one or more computer readable mediums storing instructions that, when executed by the one or more computer processors, cause the computer system to:
provide a data conversion module for deployment in a set of client nodes, the data conversion module when installed on client node configured to generate a set of components, the set of components including at least a pre-assessment tool and a data conversion model for performing one or more conversion operations on client data associated with the client node;
provide, to each client node, a base model to the client node for training by the client node using the client data;
receive, from the pre-assessment tool on each client node, statistics and abstract information describing the client data for the client node;
generate, for each client node, conversion logic based on the received statistics and abstract information on the client data for the client node, wherein the conversion logic includes instructions for converting values of the client data for one or more data fields to match a target schema for training the base model;
provide, to each client node, the conversion logic to the data conversion model of the client node; and
receive, from each client node, trained parameters of the base model from the client node, wherein the parameters of the base model are trained using converted values by executing the data conversion model to perform one or more conversion operations using the conversion logic.

16. The computer system of claim 15, wherein conversion logic generated for a first client node in the set of client nodes is different from conversion logic generated for a second client node.

17. The computer system of claim 15, wherein the conversion logic includes logic for one or a combination of data field conversion, a conversion of measurements, a value bucketing conversion, and vocabulary conversion from a first vocabulary to a second vocabulary.

18. The computer system of claim 15, the statistics and abstract information received from a client node including one or a combination of metadata on data fields of the client data or statistics on values of the client data, without exposing actual values of the client data.

19. The computer system of claim 15, the statistics and abstract information for a client node including local vocabulary used to encode one or more values in the client data, and the instructions when executed by the computer system further causing the computer system to:
generate a mapping from the local vocabulary to a standardized vocabulary; and
provide the mapping to the data conversion module.

20. The computer system of claim 15, the instructions when executed by the computer system causing the computer system to:
receive, from a client node, an indication that the statistics and abstract information for the client data has been updated;
generate updated conversion logic reflecting the changes to the statistics and abstract information; and
provide the updated conversion logic to the data conversion module of the client node.
